(19) United States
(12) Patent Application Publication
(10) Pub. No.: US 2013/0296681 A1
(43) Pub. Date: Nov. 7, 2013

(54) OPTICAL INTRAOCULAR PRESSURE MEASURING APPARATUS AND OPERATING METHOD THEREOF

(71) Applicant: CRYSTALVUE MEDICAL CORPORATION, Gueishan (TW)

(72) Inventors: William WANG, Taoyuan City (TW); Chung-Ping CHUANG, Taoyuan City (TW); Meng-Shin YEN, Taipei City (TW); Chung-Cheng CHOU, Luzhu Township (TW); Wen-Wei HUANG, Taoyuan City (TW); Chun-Nan LIN, Bade City (TW)

(73) Assignee: Crystalvue Medical Corporation, Gueishan (TW)

(21) Appl. No.: 13/886,767

(22) Filed: May 3, 2013

(30) Foreign Application Priority Data
May 4, 2012 (TW) 101116030

(51) Int. Cl. A61B 3/00; A61B 3/16

(52) U.S. Cl. CPC A61B 3/0008; A61B 3/16
USPC 600/405

(57) ABSTRACT

An optical intraocular pressure measuring apparatus includes a light source, an optical module, a pressure providing module, a deformation measuring module, and a processing module. The light source provides an incident light. The optical module divides the incident light into a first incident light and a second incident light and emits them to a reference object and an object to be detected through a first light path and a second light path, and receives a first reflected light signal from reference object and a second reflected light signal from the object to be detected respectively. The pressure providing module coupled with second light path provides a pressure to deform the object to be detected. The deformation measuring module measures the deformation of the object to be detected. The processing module processes the first reflected light signal and second reflected light signal to generate an intraocular pressure measurement result.

### BACKGROUND OF THE INVENTION

1. Field of the Invention

The invention relates to optical measurement, in particular, to a non-contact optical intraocular pressure measuring apparatus and operating method thereof.

2. Description of the Prior Art

In recent years, with the continuous development of the optical detection technology, several kinds of optical detection equipments have been developed. The optical interference technology can be widely used in human body function detection and medical diagnosis.

For example, the optical interference technology has been applied in an intraocular pressure measuring apparatus for organism. However, during the process of the current intraocular pressure measuring apparatus measuring the intraocular pressure of organism, the eye ball is easy to be vibrated by force or generates vibration itself, so that many errors occur in measurement. In addition, the measurement range of the current intraocular pressure measuring apparatus is limited, although 2-D or 3-D cornea data can be obtained by the multi-point measurements in depth direction, it needs long time to finish the measurements and the measurement results may correspond to different positions since the eye ball is in a dynamic state, and some cornea data will be lost or have errors.

### SUMMARY OF THE INVENTION

Therefore, the invention provides an optical intraocular pressure measuring apparatus and operating method thereof to solve the above-mentioned problems.

A first embodiment of the invention is an optical intraocular pressure measuring apparatus. In this embodiment, the optical intraocular pressure measuring apparatus includes a light source, an optical module, a pressure providing module, a deformation measuring module, and a processing module. The light source provides an incident light. The optical module divides the incident light into a first incident light and a second incident light and emits them to a reference object and an object to be detected through a first light path and a second light path, and receives a first reflected light signal from the reference object and a second reflected light signal from the object to be detected respectively. The pressure providing module coupled with the second light path provides a pressure to deform the object to be detected. The deformation measuring module measures the deformation of the object to be detected. The processing module processes the first reflected light signal and second reflected light signal to generate an intraocular pressure measurement result.

In an embodiment, the optical module includes a light dividing/coupling unit, a light path unit, and an image sensing unit. The light dividing/coupling unit is used for dividing the incident light into the first incident light and the second incident light, emitting the first incident light and the second incident light to the reference object and the object to be detected through the first light path and the second light path respectively, and receiving the first reflected light signal from the reference object and the second reflected light signal from the object to be detected respectively. The light path unit includes a set of lens having a lens element or a plurality of lens elements, and used for amplifying the second reflected light signal.

In an embodiment, the image sensing unit includes a 1-D or 2-D optical image sensor which is a charge-coupled device (CCD) type or a complementary metal-oxide-semiconductor (CMOS) type.

In an embodiment, the optical module provides a large-area measurement on the object to be detected by matching the light path unit and the image sensing unit, and compares the optical image of the object to be detected to determine an offset of a mark on the object to be detected, and synchronously captures the optical image of the object to be detected in depth direction to dynamically perform measurement compensation and correction.

In an embodiment, the pressure providing module includes a pneumatic component for generating a pressurized airflow to provide the pressure to deform the object to be detected, and the second light path is partially or totally shared by the pressurized airflow, the second incident light, and the second reflected light signal.

In an embodiment, the deformation measuring module includes at least one optical emission unit and at least one optical receiving unit. The at least one optical emission unit is used for emitting a first measuring light to the object to be detected before the object to be detected is deformed and emitting a second measuring light to the object to be detected after the object to be detected is deformed. The at least one optical receiving unit is used for receiving a first reflected measuring light reflected by the object to be detected before the object to be detected is deformed and receiving a second reflected measuring light reflected by the object to be detected after the object to be detected is deformed to calculate the deformation of the object to be detected.

In an embodiment, mirrors having different curvatures or materials are used as the reference object to adjust the first reflected light signal reflected by the reference object to provide a better signal-noise ratio.

A second embodiment of the invention is an optical intraocular pressure measuring apparatus optical apparatus operating method. In this embodiment, the optical intraocular pressure measuring apparatus includes a light source, an optical module, a pressure providing module, a deformation measuring module, and a processing module. The method includes steps of: (a) the light source providing an incident light; (b) the optical module dividing the incident light into a first incident light and a second incident light, and the first incident light and the second incident light being emitted to a reference object and an object to be detected through a first light path and a second light path respectively; (c) the optical module receiving a first reflected light signal from the reference object and a second reflected light signal from the object to be detected respectively; (d) the pressure providing module coupling with the second light path and providing a pressure to deform the object to be detected; (e) the deformation measuring module measuring a deformation of the object to be detected; and (f) the processing module processing the first reflected light signal and the second reflected light signal to generate an intraocular pressure measurement result.

Compared to the prior arts, the invention provides the optical intraocular pressure measuring apparatus using optical interference technology to perform non-destructive and non-contact intraocular pressure measurement, and synchronously providing large-area measurement for the area to be detected by matching the light path unit and the image sensing unit; therefore, the time needed for cornea global measurement can be largely reduced to enhance the measuring efficiency.

In addition, during the cornea measuring process of the optical intraocular pressure measuring apparatus, it can synchronously capture optical images of the cornea in depth direction to dynamically compensate the measurement offset caused by eye ball vibration.

Moreover, during the large-area measuring process of the optical intraocular pressure measuring apparatus, the offsets of the marks on the cornea can be determined by storing and comparing the optical images to trace and lock the area to be detected to prevent some cornea data from being lost or having errors.

The advantage and spirit of the invention may be understood by the following detailed descriptions together with the appended drawings.

### BRIEF DESCRIPTION OF THE APPENDED DRAWINGS

FIG. 1 illustrates a schematic figure of the optical intraocular pressure measuring apparatus in an embodiment of the invention.

FIG. 2A illustrates a detailed schematic figure of the optical intraocular pressure measuring apparatus.

FIG. 2B illustrates a schematic figure of the light path unit formed by two convex lens elements.

FIG. 2C illustrates a schematic figure of the light path unit formed by convex lens having adjustable curvature.

FIG. 3A illustrates a front view of the object (cornea) to be detected performed by a small-area measurement of the prior art and a large-area measurement of the invention.

FIG. 3B illustrates a side view of the object (cornea) to be detected performed by a small-area measurement of the prior art and a large-area measurement of the invention.

FIG. 4A and FIG. 4B illustrate the measurement performed on the object to be detected by the optical intraocular pressure measuring apparatus when the object to be detected moves a distance from the original first position to the second position.

FIG. 5A and FIG. 5B illustrate that the optical intraocular pressure measuring apparatus determines the offsets of different marks on the object (cornea) to be detected by a way of storing and comparing optical images of the object (cornea) to be detected.

FIG. 6 illustrates a flowchart of the optical intraocular pressure measuring apparatus operating method in another embodiment of the invention.

### DETAILED DESCRIPTION OF THE INVENTION

A first embodiment of the invention is an optical intraocular pressure measuring apparatus. In this embodiment, the optical intraocular pressure measuring apparatus uses optical interference technology to perform non-destructive and non-contact large-area measurement on the cornea; therefore, the measurements of the parameters (e.g., the thickness, curvature, and deformation) of the cornea can be done at the same time to obtain 2-D (surface) and 3-D (surface and vertical direction) cornea data to effectively reduce the measurement errors caused by the cornea under the dynamical state in prior arts.

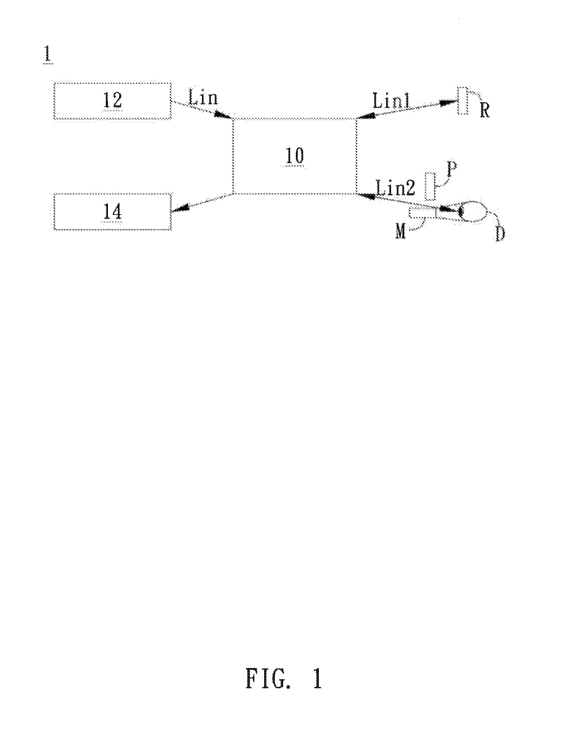

Please refer to FIG. 1. FIG. 1 illustrates a schematic figure of the optical intraocular pressure measuring apparatus in this embodiment. As shown in FIG. 1, the optical intraocular pressure measuring apparatus 1 uses optical interference technology to measure the object to be detected, such as eye ball of organism. The optical intraocular pressure measuring apparatus 1 includes a light source 12, an optical module 10, a pressure providing module P, a deformation measuring module M, and a processing module 14. Wherein, the processing module 14 is coupled to the optical module 10.

In this embodiment, the light source 12 is used to provide an incident light Lin emitting toward the optical module 10. In fact, the light source 12 can use laser to achieve low coherence interferometry (LCI). The type of the light source 12 can be a single point light source, a matrix of light sources, or an angle adjustable optical fiber light source without specific limitations.

After the optical module 10 receives the incident light Lin, the optical module 10 will divide the incident light Lin into a first incident light Lin1 and a second incident light Lin2, and the first incident light Lin1 and the second incident light Lin2 will be emitted toward a reference object R and an object to be detected D through a first light path and a second light path respectively, and the optical module 10 will also receive a first reflected light signal reflected by the reference object R and a second reflected light signal reflected by the object to be detected D respectively. The processing module 14 will process and analyze the first reflected light signal and the second reflected light signal to generate an intraocular pressure measurement result.

It should be noticed that the pressure providing module P couples the second light path LP2 between the optical module 10 and the object to be detected D and provides a pressure to deform the object to be detected D. At this time, the deformation measuring module M can measure a deformation of the object to be detected D to generate the intraocular pressure measurement result according to the deformation of the object to be detected D. The mirrors having different curvatures or materials are used as the reference object R to adjust the first reflected light signal reflected by the reference object to provide a better signal-noise ratio, but not limited to this.

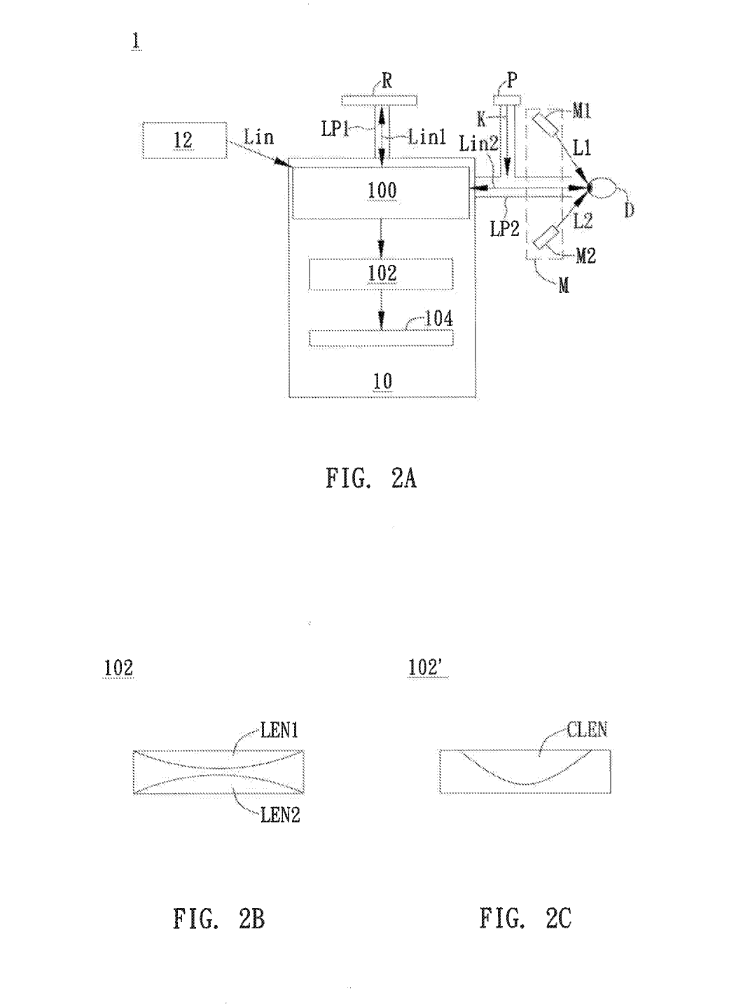

Please refer to FIG. 2. FIG. 2A illustrates a detailed schematic figure of the optical intraocular pressure measuring apparatus 1 in FIG. 1. As shown in FIG. 2A, the optical module 10 includes a light dividing/coupling unit 100, a light path unit 102, and an image sensing unit 104. The light dividing/coupling unit 100 is used for dividing the incident light Lin into the first incident light Lin1 and the second incident light Lin2, the first incident light Lin1 and the second incident light Lin2 are emitted to the reference object R and the object to be detected D through the first light path LP1 and the second light path LP2 respectively, and the light dividing/coupling unit 100 receives the first reflected light signal from the reference object R and the second reflected light signal from the object to be detected D respectively. The light path unit is used for amplifying the second reflected light signal reflected by the object to be detected D. The image sensing unit 104 is used for sensing the second reflected light signal amplified by the light path unit 102 to generate an optical image of the object to be detected D.

In practical applications, the light path unit 102 can include a set of lens having a lens element or a plurality of lens elements, for example, the light path unit 102 shown in FIG. 2B is formed by two lens elements LEN1 and LEN2; the light path unit 102′ shown in FIG. 2C is formed by a curvature adjustable lens CLEN, but not limited to this. The image sensing unit 104 can include a 1-D (strip-type) or 2-D (plane-type) optical image sensor, such as a charge-coupled device (CCD) type optical image sensor or a complementary metal-oxide-semiconductor (CMOS) type optical image sensor, but not limited to this.

In this embodiment, the pressure providing module P can be a pneumatic component for generating a pressurized airflow K to provide the pressure to deform the object to be detected D. Since the pressure providing module P is coupled with the second light path LP2, the second light path LP2 is partially or totally shared by the pressurized airflow K, the second incident light Lin2, and the second reflected light signal to enhance the measuring effect of the deformation of the object to be detected D. That is to say, there is no specific limitation to the position that the pressurized airflow K generated by the pressure providing module P enters into the second light path LP2, it depends on practical needs.

The deformation measuring module M includes at least one optical emission unit M1 and at least one optical receiving unit M2. The optical emission unit M1 is used for emitting a first measuring light L1 to the object to be detected D before the object to be detected D is deformed and emitting a second measuring light L1 to the object to be detected D after the object to be detected D is deformed. The optical receiving unit M2 is used for receiving a first reflected measuring light L2 reflected by the object to be detected D before the object to be detected D is deformed and receiving a second reflected measuring light L2 reflected by the object to be detected D after the object to be detected D is deformed to calculate the deformation of the object to be detected D according to different reflected measuring light L2 received by the object to be detected D before and after the object to be detected D. In practical applications, the deformation measuring module M can include a single optical emission unit M1 and a single optical receiving unit M2, a single optical emission unit M1 and a plurality of optical receiving units M2, a plurality of optical emission unit M1 and a single optical receiving units M2, or a plurality of optical emission unit M1 and a plurality of optical receiving units M2. It depends on practical needs.

It should be noticed that the optical intraocular pressure measuring apparatus 1 provides non-destructive and non-contact large-area measurement on the object to be detected (cornea) D by matching the light path unit 102 and the image sensing unit 104 of the optical module 10; therefore, the measurements of the parameters (e.g., the thickness, curvature, and deformation) of the object to be detected (cornea) D can be done at the same time to obtain 2-D (surface) and 3-D (surface and vertical direction) cornea data to effectively enhance the measuring efficiency of the optical intraocular pressure measuring apparatus 1.

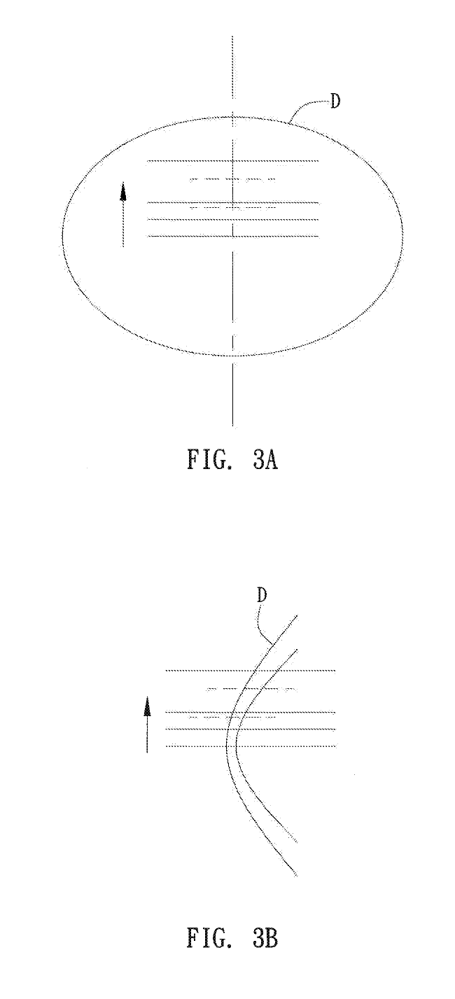

Please refer to FIG. 3A and FIG. 3B. FIG. 3A illustrates a front view of the object to be detected (cornea) D performed by a small-area measurement of the prior art and a large-area measurement of the invention. FIG. 3B illustrates a side view of the object to be detected (cornea) D performed by a small-area measurement of the prior art and a large-area measurement of the invention. As shown in FIG. 3A and FIG. 3B, the solid lines represent the small-area measurement of the prior art, and the dotted lines represent the large-area measurement of the invention.

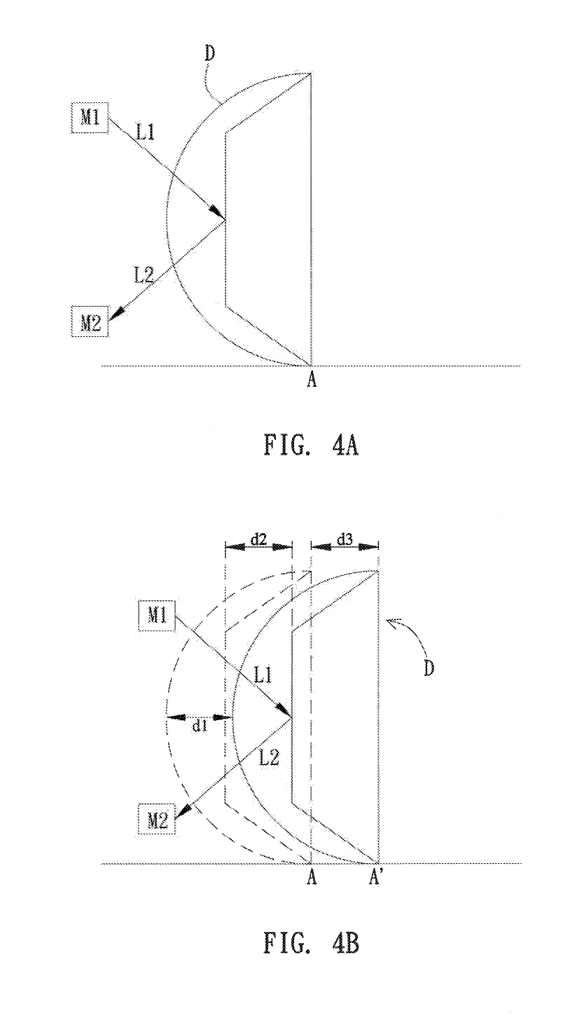

In addition, during the process of the current intraocular pressure measuring apparatus measuring the intraocular pressure of organism, the eye ball is easy to be vibrated by force or generates vibration itself, so that many errors occur in measurement. In view of this, during the deformation measuring module M of the optical intraocular pressure measuring apparatus 1 measures the deformation of the object to be detected (cornea) D, even the object to be detected (cornea) D generates unexpected vibration in depth direction (as shown in FIG. 4A and FIG. 4B, the object to be detected D horizontally moves an offset d3 from the first position A to the second position A′, and two other parts of the object to be detected D also move an offsets d1 and d2 respectively), the optical intraocular pressure measuring apparatus 1 can still use the optical receiving units M2 to receive the reflected lights reflected by the object to be detected (cornea) D at the same time to obtain optical images of the object to be detected (cornea) D in depth direction. The optical images can not only be provided to correct parameters of the thickness, curvature, and deformation of cornea, but also the dynamical image to correct the signal errors.

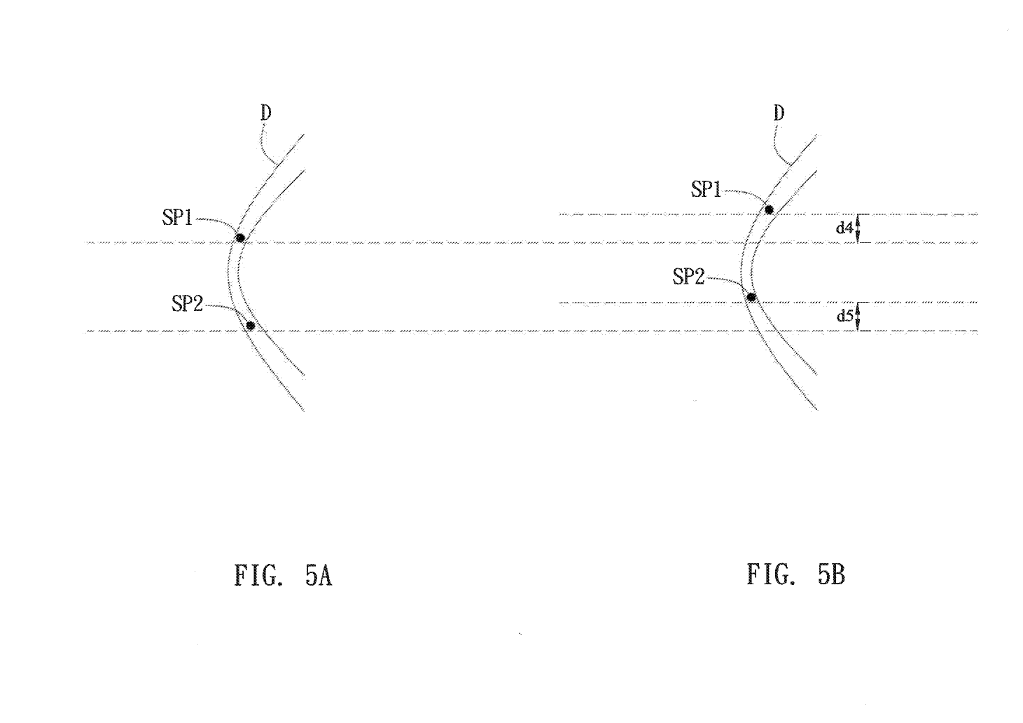

Furthermore, even the person to be detected maintains his/her eyes under a staring state, however, the measurement results may correspond to different positions since the eye ball is in a dynamic state, and some cornea data will be lost or have errors. In view of this, as shown in FIG. 5A and FIG. 5B, during the optical intraocular pressure measuring apparatus 1 performs large-area measurement on the object to be detected (cornea) D, the optical intraocular pressure measuring apparatus 1 can determine the offsets d4 and d5 of the different marks SP1 and SP2 on the object to be detected (cornea) D in vertical direction by storing and comparing optical images of the object to be detected (cornea) D. Therefore, it can effectively reduce the offsets caused by the object to be detected (cornea) D under dynamical state to prevent the cornea data from being lost or having errors.

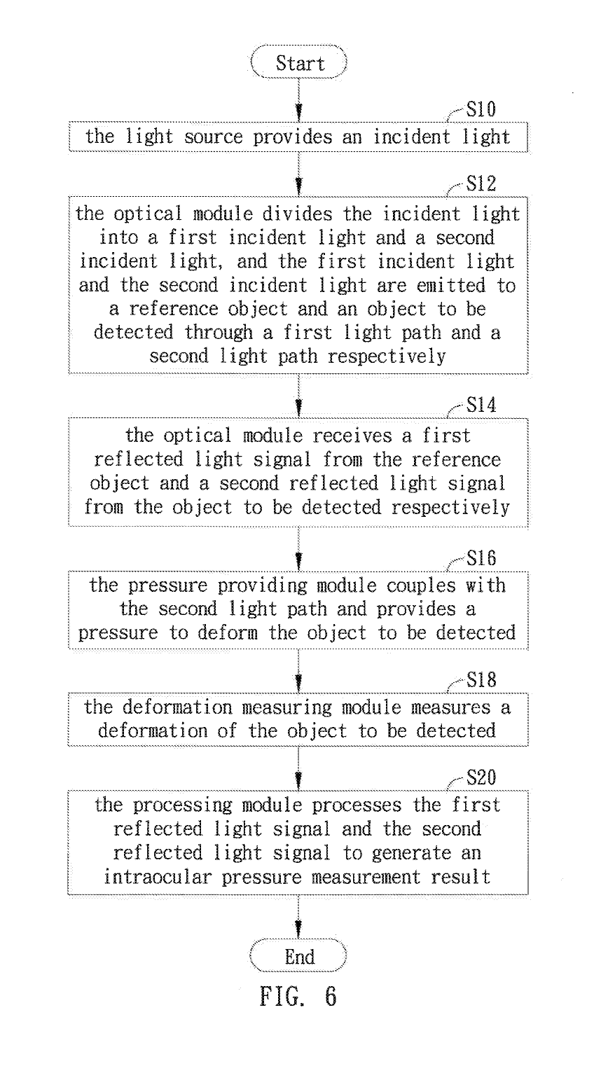

Another embodiment of the invention is an optical intraocular pressure measuring apparatus operating method. In this embodiment, the optical intraocular pressure measuring apparatus includes a light source, an optical module, a pressure providing module, a deformation measuring module, and a processing module. Please refer to FIG. 6. FIG. 6 illustrates a flowchart of the optical intraocular pressure measuring apparatus operating method in this embodiment.

As shown in FIG. 6, the optical intraocular pressure measuring apparatus operating method includes steps as follows. At first, in step S10, the light source provides an incident light. In step S12, the optical module divides the incident light into a first incident light and a second incident light, and the first incident light and the second incident light are emitted to a reference object and an object to be detected through a first light path and a second light path respectively. In step S14, the optical module receives a first reflected light signal from the reference object and a second reflected light signal from the object to be detected respectively. In step S16, the pressure providing module couples with the second light path and provides a pressure to deform the object to be detected. In step S18, the deformation measuring module measures a deformation of the object to be detected. In step S20, the processing module processes the first reflected light signal and the second reflected light signal to generate an intraocular pressure measurement result.

In fact, the optical module can further generate an optical image of the object to be detected according to the amplified second reflected light signal. The optical module can further provide a large-area measurement on the object to be detected by matching the light path unit and the image sensing unit, and compare the optical image of the object to be detected to determine an offset of a mark on the object to be detected, and synchronously capture the optical image of the object to be detected in depth direction to dynamically perform measurement compensation and correction.

In addition, the pressure providing module generates a pressurized airflow to provide the pressure to deform the object to be detected, and the second light path is partially or totally shared by the pressurized airflow, the second incident light, and the second reflected light signal. That is to say, the position that the pressurized airflow generated by the pressure providing module enters the second light path has no limitations depending on practical needs. And, mirrors having different curvatures or materials can be used as the reference object to adjust the first reflected light signal reflected by the reference object to provide a better signal-noise ratio, but not limited to this.

Compared to the prior arts, the invention provides the optical intraocular pressure measuring apparatus using optical interference technology to perform non-destructive and non-contact intraocular pressure measurement, and synchronously providing large-area measurement for the area to be detected by matching the light path unit and the image sensing unit; therefore, the time needed for cornea global measurement can be largely reduced to enhance the measuring efficiency.

In addition, during the cornea measuring process of the optical intraocular pressure measuring apparatus, it can synchronously capture optical images of the cornea in depth direction to dynamically compensate the measurement offset caused by eye ball vibration.

Moreover, during the large-area measuring process of the optical intraocular pressure measuring apparatus, the offsets of the marks on the cornea can be determined by storing and comparing the optical images to trace and lock the area to be detected to prevent some cornea data from being lost or having errors.

With the example and explanations above, the features and spirits of the invention will be hopefully well described. Those skilled in the art will readily observe that numerous modifications and alterations of the device may be made while retaining the teaching of the invention. Accordingly, the above disclosure should be construed as limited only by the metes and bounds of the appended claims.

What is claimed is:

1. An optical intraocular pressure measuring apparatus, comprising:
a light source, for providing an incident light;
an optical module, for dividing the incident light into a first incident light and a second incident light, emitting the first incident light and the second incident light to a reference object and an object to be detected through a first light path and a second light path respectively, and receiving a first reflected light signal from the reference object and a second reflected light signal from the object to be detected respectively;
a pressure providing module, coupled with the second light path, for providing a pressure to deform the object to be detected;
a deformation measuring module, for measuring a deformation of the object to be detected; and
a processing module, coupled to the optical module, for processing the first reflected light signal and the second reflected light signal to generate an intraocular pressure measurement result.

2. The optical intraocular pressure measuring apparatus of claim 1, wherein the optical module comprises:
a light dividing/coupling unit, for dividing the incident light into the first incident light and the second incident light, emitting the first incident light and the second incident light to the reference object and the object to be detected through the first light path and the second light path respectively, and receiving the first reflected light signal from the reference object and the second reflected light signal from the object to be detected respectively;
a light path unit, comprising a set of lens having a lens element or a plurality of lens elements, for amplifying the second reflected light signal; and
an image sensing unit, for sensing the second reflected light signal amplified by the light path unit to generate an optical image of the object to be detected.

3. The optical intraocular pressure measuring apparatus of claim 2, wherein the image sensing unit comprises a 1-D or 2-D optical image sensor which is a charge-coupled device (CCD) type or a complementary metal-oxide-semiconductor (CMOS) type.

4. The optical intraocular pressure measuring apparatus of claim 2, wherein the optical module provides a large-area measurement on the object to be detected by matching the light path unit and the image sensing unit, and compares the optical image of the object to be detected to determine an offset of a mark on the object to be detected, and synchronously captures the optical image of the object to be detected in depth direction to dynamically perform measurement compensation and correction.

5. The optical intraocular pressure measuring apparatus of claim 1, wherein the pressure providing module comprises a pneumatic component for generating a pressurized airflow to provide the pressure to deform the object to be detected, the second light path is partially or totally shared by the pressurized airflow, the second incident light, and the second reflected light signal.

6. The optical intraocular pressure measuring apparatus of claim 1, wherein the deformation measuring module comprises:
at least one optical emission unit, for emitting a first measuring light to the object to be detected before the object to be detected is deformed and emitting a second measuring light to the object to be detected after the object to be detected is deformed; and
at least one optical receiving unit, for receiving a first reflected measuring light reflected by the object to be detected before the object to be detected is deformed and receiving a second reflected measuring light reflected by the object to be detected after the object to be detected is deformed to calculate the deformation of the object to be detected.

7. The optical intraocular pressure measuring apparatus of claim 1, wherein mirrors having different curvatures or materials are used as the reference object to adjust the first reflected light signal reflected by the reference object to provide a better signal-noise ratio.

8. A method of operating an optical intraocular pressure measuring apparatus, the optical apparatus comprising a light source, an optical module, a pressure providing module, a deformation measuring module, and a processing module, the method comprising steps of:
(a) the light source providing an incident light;
(b) the optical module dividing the incident light into a first incident light and a second incident light, and the first incident light and the second incident light being emitted to a reference object and an object to be detected through a first light path and a second light path respectively;
(c) the optical module receiving a first reflected light signal from the reference object and a second reflected light signal from the object to be detected respectively;
(d) the pressure providing module coupling with the second light path and providing a pressure to deform the object to be detected;
(e) the deformation measuring module measuring a deformation of the object to be detected; and
(f) the processing module processing the first reflected light signal and the second reflected light signal to generate an intraocular pressure measurement result.

9. The method of claim 8, wherein the optical module further generates an optical image of the object to be detected according to the amplified second reflected light signal, and the optical module further provides a large-area measurement on the object to be detected by matching the light path unit and the image sensing unit, and compares the optical image of the object to be detected to determine an offset of a mark on the object to be detected, and synchronously captures the optical image of the object to be detected in depth direction to dynamically perform measurement compensation and correction.

10. The method of claim 8, wherein the pressure providing module generates a pressurized airflow to provide the pressure to deform the object to be detected, and the second light path is partially or totally shared by the pressurized airflow, the second incident light, and the second reflected light signal.
